(19) United States
(12) Patent Application Publication
(10) Pub. No.: US 2003/0054353 A1
(43) Pub. Date: Mar. 20, 2003

(54) High throughput method for discovery of gene clusters

(75) Inventors: Chris M. Farnet, Outremont (CA); Emmanuel Zazopoulos, Outremont (CA); Alfredo Staffa, St-Leonard (CA)

Correspondence Address: ECOPIA BIOSCIENCES INC., 7290 FREDERICK-BANTING, SAINT-LAURENT, QC, H4S 2A1, CA

(73) Assignee: ECOPIA BIOSCIENCES INC

(21) Appl. No.: 09/910,813

(22) Filed: Jul. 24, 2001

(51) Int. Cl. C12Q 1/68; G06F 19/00; G01N 33/48; G01N 33/50

(52) U.S. Cl. 435/6; 702/20

(57) ABSTRACT

A method for identifying gene cluster is disclosed. The method may be used for identifying gene clusters involved in the biosynthesis of natural products. A small insert library of DNA fragments of genomic DNA and a large insert library of DNA fragments of genomic DNA are prepared. Fragments in the small insert library are sequenced and compared by homology comparison under computer control to a database containing genes, gene fragments or proteins known to be involved in the biosynthesis of microbial natural products. Fragments having similar structure to genes, gene fragments or proteins known to be involved in the biosynthesis of naturally occurring metabolites are used as probes to screen the large insert library of genomic DNA to detect gene clusters involved in the biosynthesis of microbial natural products.

### FIELD OF INVENTION

The invention relates to the fields of microbiology and genomics, and more particularly to a high-throughput method for discovery of gene clusters.

### BACKGROUND

The method of the present invention allows rapid discovery of gene clusters involved in metabolic pathways or other processes without having to sequence the entire genome.

Microbial genes whose products act in a coordinated fashion, for example a biosynthetic pathway, are often arranged in close physical proximity to one another in the organism's genome. Such genes are said to form a gene cluster. Gene clusters are associated with a variety of metabolic pathways, notably the biosynthesis of microbial natural products. Gene clusters may also provide resistance to therapeutic drugs. (See for example, Schouten et al., Molecular analysis of Tn1546-like elements in vancomycin-resistant enterococci isolated from patients in Europe shows geographic transposon type clustering, Antimicrob Agents Chemother, 45(3):986-9). Gene clusters are also associated with pathogenicity islands from various organisms. (See for example, Kuroda et al., Whole genome sequencing of methicillin-resistant Staphylococcus aureus, Lancet, 357(9264):1225-40; Carniel E., The Yersinia high-pathogenicity island: an iron-uptake island, Microbes Infect. 3(7):561-9; Nicholls et al., Identification of a novel genetic locus that is required for in vitro adhesion of a clinical isolate of enterohaemorrhagic Escherichia coli to epithelial cells, Mol. Microbiol 35(2):275-88). Gene clusters are also responsible for catabolic pathways. (See for example, Velasco et al., Genetic and functional analysis of the styrene catabolic cluster of Pseudomonas sp. strain Y2, J. of Bacteriology, 180(5):1063-1071; Buchan et al., Key aromatic-ring-cleaving enzyme, protocatechuate 3,4-dioxygenase, in the ecologically important marine Roseobacter lineage, Appl. and Env. Microbiol., 66(11): 4662-4672; Masai et al., Genetic and biochemical characterization of a 2-Pyrone-4,6-dicarboxylic acid hydrolase involved in the protocatechuate 4,5-cleavage pathway of Sphingomonas paucimobilis SYK-6, J. of Bacteriology, 181(1):55-62; Ferrandez et al., Catabolism of phenylacetic acid in Escherichia coli, J. of Biological Chemistry, 273(40), 25974-25986).

Bioactive small molecules or natural products produced by microbial secondary metabolism are a prime example of compounds produced by gene clusters. The genes encoding natural product biosynthetic pathways in both prokaryotes and eukaryotes are clustered together on gene clusters. These gene clusters, which typically range in size from 50 kilobase pairs (kbp) to 200 kbp, also usually contain resistance genes and pathway-specific regulatory genes. See, for example, Cole S. T. and Saint Girons I., Bacterial genomics, FEMS Microbial Rev., 14(2):139-60.

Gene clusters in general are of significant interest in various fields. For example, gene clusters such as the Tn1546-like elements that are responsible for the spread of vancomycin resistance in clinical isolates of enterococci are of great interest to the medical field. The rapid identification of such clusters allows a better understanding of the spread and mechanisms of action of such gene clusters. Gene clusters for catabolic pathways are of interest in the field of bioremediation for the breakdown of toxic agents from contaminated environments and in the field of chemical engineering for the generation of economically valuable molecules from common, inexpensive materials. Gene clusters known as pathogenicity islands render otherwise harmless bacteria to highly pathogenic threats. For example, E. coli 0157 is a clinically important and often lethal pathogen that differs in part from the non-pathogenic E. coli K12 in that the former contains pathogenicity islands. Identification of such pathogenicity islands are of great importance to the medical field.

Natural product biosynthetic gene clusters are of significant interest in the field of combinatorial biosynthesis and metabolic engineering. It is now commonplace to make novel molecules by genetic engineering of natural product biosynthetic genes and there are many different approaches to generating novel metabolites. Novel pathways may be created by rearranging the genes in the gene cluster or by combining one or more of the genes from the gene cluster with other genes. For example, genes encoding enzymes catalyzing decorating reactions such as hydroxylation, methylation, acetylation, oxidation, reduction etc. of known natural products can be inserted into or deleted from a pathway gene cluster to effect production of unnatural metabolites. Alternatively, novel products may be generated by gene addition, gene knockout, gene substitution, or site-specific modification of genes encoding the enzymes that catalyze decorating reactions. Novel products can also be generated in surrogate host organisms from heterologous libraries created from prokaryotic or eukaryotic gene clusters. Individual genes drawn from microbial gene clusters may be used as biocatalysts either in cell-free systems such as a purified or partially purified enzymatic activities or in an appropriate heterologous expression host. Improved methods to rapidly discover gene clusters involved in the biosynthesis of microbial natural products expands the repertoire of genes available for use in combinatorial biosynthesis and as biocatalysts.

The emergence of bacteria resistant to multiple antibiotics has led to renewed interest in isolating variants of known antibiotics and novel antibiotics, and also in identifying new genes and gene products that could serve as new targets for new or existing antibiotics. However, methods for natural product discovery have faced many challenges. Discovery efforts that focus on plant derived natural products are hampered by limited source material, typically low concentrations of active metabolite, difficulty extracting useful quantities of the natural product produced, and the fact that many secondary metabolic biosynthetic loci are expressed only under particular growth conditions (for example, pathogen infestation) that are poorly understood and may be difficult to reproduce experimentally. Discovery efforts that focus on microbial derived natural products are hampered by difficulties in cultivating the microbes; indeed most microbes cannot be cultivated. In addition, many cultivated microorganisms are not amenable to fermentation. Furthermore many secondary biosynthetic loci are not expressed to detectable levels under in vitro conditions. Furthermore, natural products produced under in vitro conditions often vary according to the growth conditions, e.g. nutrients provided, and may not be representative of the full biosynthetic potential of the microorganism. Thus, there is a need for improved methods for discovery of gene clusters involved in the biosynthesis of natural products.

There also exist limitations in current methods to clone natural product biosynthetic loci from known producer microorganisms. Many known methods are time consuming and require genetic tools that that are not available for most microorganisms, for example mutagenesis followed by complementation with genomic libraries, or transposon-tagging. Other known methods are time consuming and have limited chance of success, for example heterologous expression, and heterologous expression of resistance in order to clone linked genes. Other known methods require prior knowledge of the structural class of the natural product and DNA sequence information from a related biosynthetic locus, for example PCR amplification or cloning by hybridization analysis. Thus, it is desirable to obtain method of rapidly identifying and cloning from microbial genomes the complete genetic locus responsible for the biosynthesis of natural products having antibiotic activity.

The clustering together of biosynthetic and resistance genes encoding natural product biosynthetic pathways has allowed for several methods for searching for natural product biosynthesis gene clusters. For example, resistance has been used as a selective probe for clones (Walczak et al., Nonactin biosynthesis: the potential nonactin biosynthesis gene cluster contains type II polyketide synthase-like genes, FEMS Microbiol. Lett. 183(2000), 171-175), or selection based on the activity of a single enzyme within the cluster (Jones and Hopwood, 1984, Molecular cloning and expression of the phenoxazinone synthase gene from Streptomyces antibioticus, J. Biol. Chem. 259,14151-14157).

Natural product biosynthetic gene clusters have also been identified using hybridization to highly-conserved heterologous genes. Hybridization-based approaches have been used in relation to Type I polyketide ketosynthase domains (Beyer et al., 1999, Metabolic diversity in myxobacteria: Identification of the myxalamid and the stigmatellin biosynthetic gene cluster of Stigmatella aurantiaca Sg a15 and a combined polyketide-(poly)peptide gene cluster from the epothilone producing strain Sorangium cellulosum So ce90, Biochim. Biophys. Acta, 1445, 185-195; Suwa et al., 2000, Identification of two polyketide synthase gene clusters on the linear plasmid pSLA2-L in Streptomyces rochei. Gene 246, 123-131) or Type II polyketide ketosynthase domains (Malpartida et al., 1987, Homology between Streptomyces genes coding for synthesis of different polyketides used to clone antibiotic biosynthetic genes, Nature, 325, 818-821; Lombo et al., 1996, Characterization of Streptomyces argillaceus genes encoding a polyketide synthase involved in the biosynthesis of the antitumor mithramycin, Gene, 172, 87-91), non-ribosomal peptide synthetase (NRPS) domains (Beyer et al., supra), or other highly conserved natural product biosynthesis genes (Steffensky et al, 2000, Identification of the novobiocin biosynthetic gene cluster of Streptomyces spheroids NCIB 11891, Antimicrob. Agents Chemother. 44, 1214-1222). However, these and other hybridization methods are often cluster-specific, and may not have broad application to smaller gene clusters or non-modular gene clusters. In addition, many of these methods are labor-intensive, and involve sequencing significant amounts of DNA encoding genes that are not involved in the biosynthesis of a natural product. Because probes or primers are often imperfect, natural product gene clusters may be missed. Furthermore, probes or primers may not reveal the natural product biosynthetic loci sought as organisms often contain multiple natural product biosynthetic loci.

Advances in gene detection and sequencing methods have improved accuracy of hybridization-based approaches that involve sequence comparison to databases of known sequences. As of April 1999, the sequences for more than 150 different gene clusters encoding natural product biosynthesis pathways have been placed into the public databases (Strohl W. R., Biochemical Engineering of Natural Product Biosynthesis Pathways, Metabolic Engineering 3(2000), 4-14). With recent advances in sequencing brought about by automatic sequencers, such as the ABI Prism 3700 Genetic Analyzer, which is capable of handling a throughput of over half a billion bases per day, the number of natural product gene clusters available in public databases is expected to grow exponentially. This wealth of information facilitates increasingly accurate and informative bioinformatic analyses.

There is a continuing need for high throughput methods for identification of all gene clusters in a microbial genome. There is also a need for methods for detecting natural product loci in a genome with minimal DNA sequencing, and in particular minimal sequencing of DNA encoding genes for primary metabolism. There is also a need for improved methods for detecting the biosynthetic loci for secondary metabolic pathways in an organism without having to sequence the entire microbial genome of the organism. There is also a need for improved genomics-based methods for detecting gene clusters responsible for the biosynthesis of natural products in microbial organisms, which methods are rapid, use less reagents, and are less labor-intensive.

### SUMMARY OF THE INVENTION

A genomics based method to rapidly search through the genome of a microorganism in order to discover genes clusters without having to sequence entire genome has been developed. The method can be used to detect any cluster of genes that act together in a coordinated manner and are clustered together on a chromosome. In one embodiment, the method may be used to detect a gene cluster involved in the synthesis of a natural product. In another embodiment, the method may be used to detect a gene cluster involved in a catabolic pathway such as the degradation of phenolic compounds. In yet another embodiment, the method may be used to detect a gene cluster for a pathogenicity island from an organism. In yet another embodiment, the method may be used to detect a gene cluster that confers resistance to a natural product.

The invention is not limited to gene clusters having a particular structure or sequence pattern, for example modular Type I polyketide synthase genes, but rather may be used to identify a wide variety of structurally diverse gene clusters, including but not limited to those responsible for the biosynthesis of natural products such as orthosomycins, glycosylated lipodepsipeptides, and benzodiazepine antibiotics. The invention may also be used to identify polyketide synthase genes.

Discovery of gene clusters using the present invention is not dependent upon expression of the natural product. Thus, it is possible to discover new natural products that are not expressed at a level sufficient for detection using more traditional approaches. In one embodiment, the organism is a known producer of a natural product, although the gene cluster responsible for production of the known natural product is unknown. In another embodiment, the organism is known to produce a particular natural product or multiple natural products but also contains a further gene cluster for the biosynthesis of natural products undetected by traditional methods. In another embodiment the organism is not known to produce a natural product. The genome of many microorganisms contains multiple natural product biosynthetic loci and the present invention may be used to detect all natural product biosynthetic loci present in an organism's genome while minimizing the amount of DNA sequencing required. In addition, the methods of the present invention do not require the cultivation, growth or fermentation of organisms.

The invention involves the cloning of gene clusters by a method that combines random DNA sequencing followed by computer analysis of the DNA sequence. The method further involves the use of spot or shotgun DNA sequencing of a genome using a library of small clones with concomitant mapping of them to cosmid libraries.

Thus, in one aspect, the invention provides a method for detecting genes which act together in a coordinated manner and are clustered together on a chromosome comprising: (a) preparing from isolated genomic DNA a small insert library of DNA fragments of the genomic DNA and a large insert library of DNA fragments of the genomic DNA; (b) determining the DNA sequence of at least part of the fragments in the small insert library to form a plurality of Gene Sequence Tags (GSTs); (c) comparing, under computer control, the DNA sequence of the GSTs or the amino acid sequences corresponding to the DNA sequence of the GSTs with a database containing genes, gene fragments or DNA, or amino acid sequences known to be part of a cluster of genes that act together in a coordinated manner to identify GSTs that have similar structure to genes, gene fragments or amino acid sequences known to be part of a cluster of genes that act together in a coordinated manner; and (d) using the GSTs having similar structure to genes, gene fragments, DNA or amino acid sequences known to be part of a cluster of genes that act together in a coordinated manner as probes to screen the large insert library of genomic DNA to detect genes which act together in a coordinated manner and are clustered together on a chromosome.

In another aspect, the invention provides a method for identifying genes and gene clusters involved in the biosynthesis of microbial natural products comprising: (a) isolating genomic DNA from an organism or natural environment; (b) preparing a small insert library of DNA fragments, for example of about 1.5 kbp to about 10 kbp, of the genomic DNA, and a large insert library of DNA fragments of a significant size, for example of about 30 kbp to 50 kbp of the genomic DNA in the case of cosmid libraries; (c) determining the sequence of at least part of the fragments in the small insert library to form a plurality of gene sequence tags (GSTs); (d) comparing, under computer control, the sequences of the GSTs with a database containing genes, gene fragments, DNA or amino acid sequences known to be involved in the biosynthesis of microbial natural products to identify by sequence homology the GSTs that have a similar structure to genes, gene fragments or amino acid sequences known to be involved in the biosynthesis of microbial natural products; and (e) using the GSTs having similar structure to genes, gene fragments, DNA or amino acid sequences known to be involved in the biosynthesis of microbial natural products as probes to screen the large insert library of genomic DNA to detect gene clusters involved in the biosynthesis of microbial natural product.

In yet another embodiment, the invention provides a method of cloning gene clusters wherein, following the steps in the methods described above, the DNA sequence of the large insert genomic DNA detected in steps (d) or (e) as outlined above is determined.

Although the invention requires the sequencing of hundreds of fragments of genomic DNA, the investment is small when compared to sequencing the entire genome of a microorganism.

Other objects, features and advantages of the present invention will become apparent from the following detailed description. It should be understood, however that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a schematic view of a method for discovery of a gene cluster according to one embodiment of the invention.

FIG. 2 illustrates construction of a small insert library and a large insert library according to the method of FIG. 1.

FIG. 3 illustrates selection of Gene Sequence Tags (GSTs) from the small insert library for use of probes for screening the large insert library according to the method of FIG. 1.

FIG. 4 illustrates identification and cloning of the gene cluster from the large-insert library according to the method of FIG. 1.

### DETAILED DESCRIPTION OF THE INVENTION

The present invention provides an efficient strategy for identifying genomic sequences harboring a target gene cluster.

The meaning of gene cluster, as used in the specification, refers to any group of two or more genes that act together in a coordinated manner and that are clustered together on a chromosome. The meaning of gene cluster is not restricted to or associated with any particular type of metabolic pathway. Rather, the target gene cluster of the invention may be associated with a wide range of metabolic pathways or cellular processes including, but not limited to, the biosynthesis of natural products, the degradation of a compound, conferring resistance to therapeutic drugs, or pathogenicity islands from various organisms.

The meaning of genome extends to all DNA contained within an organism, including naturally occurring plasmids or other episomal DNA, or in the case of eukaryotes, compartmentalized DNA.

The approach involves the generation of two random DNA libraries from genomic DNA of a microorganism of interest, namely a small insert library and a large insert library. The small insert library serves as a genomic sampling library. Probes derived from sequences obtained from the small insert library are used to identify and isolate from the large insert library significantly larger genomic DNA fragments that include the probe together with its flanking sequences, and genes of the target gene cluster. The small insert library is formed of a population of randomly generated fragments so as to provide an adequate sampling of the entire DNA contained within a microorganism. Advantageously, the population includes fragments of all biosynthetic loci in the genome. Fragments from the small insert library are sequenced to provide Gene Sequence Tags (GSTs). The GSTs that correspond to fragments of the target gene cluster by homology comparison with a database are used as probes to identify the large insert clone(s) containing the genes that form the target gene cluster.

The genomic DNA may be derived from any prokaryotic or eukaryotic microorganism known or suspected to contain a gene cluster. The genomic DNA may be drawn from a population of uncultured microorganisms found in their natural habitat or environment or from biomass, thereby avoiding problems associated with cultivation and fermentation of microbes. The genomic DNA may also be derived from cultured microorganisms, either mixed or purified. A preferred source of the genomic DNA is microorganisms, such as bacteria and fungi. Bacterial species suitable for use in the method include substantially all bacterial species, both animal- and plant-pathogenic and nonpathogenic. Preferred microorganisms for the purpose of identifying natural product biosynthesis clusters include but are not limited to bacteria of the order Actinomycetales. Preferred genera of Actinomycetes include Nocardia, Geodermatophilus, Actinoplanes, Micromonospora, Nocardioides, Saccharothrix, Amycolatopsis, Kutzneria, Saccharomonospora, Saccharopolyspora, Kitasatospora, Strepmyces, Microbispora, Streptosporangium, Actinomadura. The taxonomy of actinomycetes is complex and reference is made to Goodfellow (1989) Suprageneric classification of actinomycetes, Bergey's Manual of Systematic Bacteriology, Vol. 4, Williams and Wilkins, Baltimore, pp 2322-2339, and to Embley and Stackebrandt, (1994), The molecular phylogeny and systematics of the actinomycetes, Annu. Rev. Microbiol., 48, 257-289 for genera that may also be used with the present invention. One skilled in the art would understand that the preferred source of DNA will depend on the target gene cluster; e.g., actinomycetes and bacilli for natural products, pseudomonads for catabolic pathways, etc.

The genomic DNA can be isolated from samples using various techniques well known in the art (Nucleic Acids in the Environment Methods & Applications, J. T. Trevors, D. D. van Elasas, Pringer Laboratory, 1995). Preferably, the genomic DNA obtained will be of high molecular weight and free of enzyme inhibitors and other contaminants. In a preferred embodiment, the size of the genomic DNA is of a molecular weight higher than 80 kb. The genomic DNA is employed to produce two random recombinant DNA libraries. The recombinant DNA library may be prepared without prescreening the organism or population of organisms, cultured or not, for the presence of the target gene cluster. The genomic DNA fragments may be generated and subcloned into an appropriate cloning vector by a variety of procedures. Ideally, the genomic DNA fragments will be as random as possible. Mechanical shearing methods such as sonication, nebulization and the like, or passage through a fine needle with manual pressure are preferred methods, however enzymatic methods such as partial digestion with a frequently cutting restriction enzyme (for example Sau3Al or TaqI) and other methods can also be employed. When a mechanical shearing method is employed, the ends of such fragments may be “repaired” or blunted to generate uniform ends that can be enzymatically ligated to the appropriate restriction site(s) of the vector either directly or with the use of DNA linkers. Smaller inserts are preferentially cloned.

Any conventional cloning vector, suitable for genomic DNA libraries, may be used including phage-derived, plasmids, cosmids, phosmids, Bacterial Artificial Chromosomes (BACs), and Yeast Artificial Chromosomes (YACs). One skilled in the art will select an appropriate cloning vectors based on the circumstances, e.g. typical plasmid cloning range of 0.1 to 10 kbp, typical cosmid cloning range 30 to 50 kbp, typical BAC cloning range 75-300 kbp etc. In general, the DNA sequence is inserted into an appropriate restriction endonuclease site(s) on the cloning vector by procedures known in the art. Such procedures and others are deemed to be within the scope of those skilled in the art.

A small insert library of relatively short genomic DNA fragments is constructed. DNA fragments forming the small insert library are cloned into an appropriate vector and serve as a source for genetic sampling. One suitable vector that may be used to prepare the small insert library is the pBluescript II cloning vector (Stratagene). Other suitable vectors include but are not limited to pUC19 and related vectors, Lambda vectors, M13 cloning vectors, pBR322 and related vectors.

Advantageously, the small insert library size, i.e. the number of individual clones, is as random as possible and is large enough to provide an adequate representation of the DNA contained within the microorganism of interest. By estimating size of the target gene cluster and the size of the genome, a preferred small library size may be determined. For example, the frequency of sequences containing genes from secondary metabolic pathways producing natural products in the small insert library reflects their occurrence in the genome. If the microorganism has one or more naturally occurring plasmids of moderate to high copy number, or is a microorganism whose genome is segmented in a non proportional fashion, the resulting small insert library will reflect this disproportionality. To overcome any bias that may arise due to such genetic disproportionality, a larger number of small insert clones may have to be processes and the size of the large insert library may likewise have to be increased under such circumstances. Alternatively, the chromosomal DNA may be purified by methods known in the art to overcome problems due to a high copy number of plasmids. In any event, the number of cloned DNA fragments in the short insert library or the library size must provide a reasonable probability that genes from the target gene cluster will be found in the representative fragments forming the short insert library. If it is known what experimental conditions allow for the expression of a gene or enzymatic activity believed to belong to a target gene cluster, the small insert library may be enriched for clones containing DNA fragments thereof by activity selection or screening.

Preferably, the size of the DNA fragments forming the short insert sampling library will be substantially uniform. The actual size of the DNA fragments in the short insert library may vary, but the size must be of a length to provide sufficient sequence data to identify a fragment as part of the target gene cluster. In one embodiment of the invention, the size of the DNA fragments in the short insert library is about 1.5 kpb to about 10 kbp, in a preferred embodiment the size of the DNA fragments is about 1.5 kbp to about 5 kbp, in a more preferred embodiment the size of the DNA fragments is about 1.5 kpb to about 3 kbp. Since the current sequencing technology can routinely provide sequence information, referred to herein as a “read”, of up to 700 bp, and sequencing can be carried out with primers flanking both sides of the insert, it is advantageous that the insert be at least the length of two reads so that each read yields different sequence data. The use of larger inserts increases the probability of obtaining intact genes together with required regulatory sequences that may be expressed in the cloning host, especially if the cloning host is closely related to the organism from which the genomic DNA was isolated. This may not be desirable as this may skew the population towards non-toxic or non-detrimental DNA fragments or beneficial DNA fragments.

A large insert library of relatively long DNA fragments is also constructed. The DNA fragments forming the large insert library are cloned into an appropriate vector and serve as a screening library from which the target gene cluster(s) is/are obtained. Suitable vector systems for use in preparing the large insert library include but are not restricted to Lambda vectors such as Lambda DASH II, cosmid vectors such as pWE15 or SuperCos-1, P1 cloning vectors such as pAd10sacBII, fosmid vectors such as pFos1, Bacterial Artificial Chromosome (BAC) vectors such as pBeloBAC11, and Yeast Artificial Chromosomes (YAC) vectors such as pYAC4. The vector is selected to be stably propagated in an appropriate host. It is noted that the short insert library and the large insert library need not be done in the same host organism, i.e., E. coli, Bacillus, Saccharomyces cerevisiae, human cell lines, etc.

Preferably, the size of the genomic DNA fragments in the large insert library will be substantially uniform. The size of the genomic DNA fragments in the large insert library will vary widely depending on the vector system used. In the case where a cosmid vector system is employed, the size of the DNA fragment in the large-insert library is about 30 kbp to about 50 kbp.

Where the genomic DNA is isolated from a purified organism, an appropriate number of the large insert clones is one that allows several-fold coverage of the genome of interest. Where the DNA is isolated from a mixed population of organisms, the number of large insert clones should preferably be larger so as to maximize the probability to find overlapping clones.

Short lengths of DNA from either end of cloned inserts in the short insert library are sequenced using a forward primer (F) or a reverse primer (R) to provide a plurality of Gene Sequence Tags (GSTs). However, if the large insert library includes a significant number of clones and the DNA sequencing technology reproducibly yields adequate sequence information from the ends of such inserts, the GSTs can be generated from the large insert library. In such a case, the ability to sequence a significant number of ends from a large insert library can substitute for sequencing of the small insert library. In a preferred embodiment a GST is produced from each of the cloned inserts in the short insert library. The length of the GST sequence will depend on the sequencing technology used but typically ranges from about 300 bp with a traditional (manual) DNA sequencing apparatus up to about 700 bp or more with an automated DNA sequencer such as an a 3700 ABI capillary electrophoresis DNA sequencer (applied Biosystems). In one embodiment the GSTs are about 700 base pairs in length.

The sequence of each GST is provided in computer readable form for in silico screening of a database containing genes, gene fragments or DNA known to be involved in the target gene cluster. In one embodiment the in silico screening is based on the nucleic acid sequence of the GSTs. In a preferred embodiment, the nucleic acid sequence of the GST is translated to its corresponding amino acid sequence, and the in silico screening is based on the amino acid sequence of the GSTs against a database containing proteins or protein fragments known to be involved in the target gene cluster. Advantageously, translation of the nucleic acid sequence of the GSTs to their corresponding amino acid sequence or of a database of genes, gene fragments and DNA to the corresponding amino acid sequences is computer-assisted.

The nucleic acid sequence or the amino acid sequence of the GSTs, in computer readable form, is compared under computer control using publicly available bioinformatics tools such as BLAST, Prodom, Clustal, etc. to a DNA or protein database containing genes, gene fragments, or clusters of genes, or their corresponding protein products known to be involved in the target gene cluster. The database may be a public gene database such as GenBank, EMBL, or a private database. A preferred database for the identification of natural product biosynthetic genes is the Decipher™ database of microbial genes, available on a subscription basis from Ecopia BioSciences Inc., St.-Laurent, Quebec.

Advantageously, the reference database used for homology comparison contains at least one or preferably multiple homologues of one or more genes of the target gene cluster. A homologous amino acid sequence is one that differs from an amino acid sequence by one or more conservative amino acid substitutions. Such a sequence encompasses allelic variants, as well as sequences containing deletions or insertions that retain the functional characteristics of the polypeptide. Homologous amino acid sequences include sequences that are identical or substantially identical to the amino acid sequence. By amino acid sequence substantially identical is meant a sequence that differs from the sequence of reference by a majority of conservative amino acid substitutions. Conservative amino acid substitutions are substitutions among amino acids of the same class. These classes include, for example, amino acids having uncharged polar side chains, such as asparagine, glutamine, serine, threonine, and tyrosine; amino acids having basic side chains, such as lysine, arginine, and histidine; amino acids having acidic side chains, such as aspartic acid and glutamic acid; and amino acids having nonpolar side chains, such as glycine, alanine, valine, leucine, isoleucine, proline, phenylalanine, methionine, trytophan, and cysteine.

Homology comparison of the GSTs and the sequences in the database may be assessed by % identity or by E value. The E value relates the expected number of chance alignments with an alignment score at least equal to the observed alignment score. An E value of 0.00 indicates a perfect homolog. The E values are calculated as described in Altschul et al. J. Mol. Biol., October 5; 215(3) 403-10, the teachings of which are incorporated herein by reference. The E value assists in the determination of whether two sequences display sufficient similarity to justify an inference of homology. An E value of 10⁻¹⁰ will generally be indicative of two proteins that are significantly related to one another, an E value of 10⁻⁵ being especially significant. However the length and accuracy of the sequenced being compared with the database will strongly influence the value of E considered significant. The use of a filter to mask stretches of low complexity or highly biased amino acid sequences can be used to increase the specificity of homology comparisons.

Comparison of sequences may also be assessed by clustal alignments showing conserved positions between the GSTs and the sequences of the database. In this manner, GSTs likely to belong to genes involved in the target gene cluster are identified. Amino acid sequences are aligned to maximize identity. Gaps may be artificial introduced into the sequence to attain proper alignment. Once the optimal alignment has been set up, the degree of homology is established by recording all of the positions in which the amino acid of both sequences are identical, relative to the total number of positions.

Clones that contain a GST that have a similar primary amino acid sequence based on homology comparison with gene fragments known to be involved in the target gene cluster are sequenced from the short insert library. In a preferred embodiment, the DNA clone from the small insert library that corresponds to a GST of interest can be sequenced from the other end using a universal reverse primer and analyzed for homology to the reference database. Sequencing at the opposite other end of the insert from which the GST was derived identifies clones whose inserts contain GSTs that correspond to gene fragments known to be involved in the target gene cluster at both ends of a single short insert. Insert clones that display homology to the target gene cluster at both ends are likely to contain sequences from the target gene cluster. Identification of clones having homology to the target gene cluster requires the presence of characterized homologues in the reference database.

The GSTs that correspond to genes or gene fragments known to be involved in the target gene cluster are used as or used to derive hybridization probes to isolate the corresponding DNA fragments in the large insert library by standard hybridization procedures on high density array matrices such as nylon membranes or DNA microchips. Such hybridization probes can be nucleic acids, DNA or RNA, containing a sequence from the cloned DNA fragment, in full or in part, that is labeled either with a radioisotope such as ³²P or with a non-radioactive detection system such as digoxygenin (Roche). With organisms whose genome is highly biased in that it is highly GC-rich or AT-rich, larger probes can lead to non-specific hybridization or background. Therefore, for GC-rich organisms such as actinomycetes relatively short oligonucleotide probes of approximately 20 nucleotides are preferred over longer PCR-amplified fragments. In the event that the desired gene cluster extends beyond the boundaries of a large insert clone or a series of overlapping large insert clones, the DNA sequence at these boundaries can be used to design additional probes which can be used in another round of hybridization to identify other overlapping large insert clones. This second round of hybridization can be performed at any stage of detection and cloning of the target gene cluster from the large insert library or final assembly of the target gene cluster.

The insert of the large insert clone is entirely sequenced by any method known in the art. In one embodiment, the insert of the large insert clone is sequenced by a shotgun DNA sequencing technique. In other embodiments, the insert of the large insert clone is sequenced by a technique selected from a subcloning technique, a primer walking technique, and a nested deletion technique.

The cloned sequences are then assembled and the open reading frames are identified using appropriate methods known to one skilled in the art. These methods or criteria for gene identification can vary depending on the nature of the organism from which the genomic DNA was isolated. Overlapping large insert clones can be assembled together using computer algorithms to generate a large, contiguous DNA contig sequence or multiple DNA contig sequences that are separated by relatively small gaps. One skilled in the art can then analyze these contigs of DNA sequence using bioinformatics tools to identify the open reading frames and regulatory sequences. The sequences are assembled into the target gene cluster by additional computer analysis. The sequences of the DNA contigs and the proteins which they are predicted to encode can then be submitted to appropriate databases.

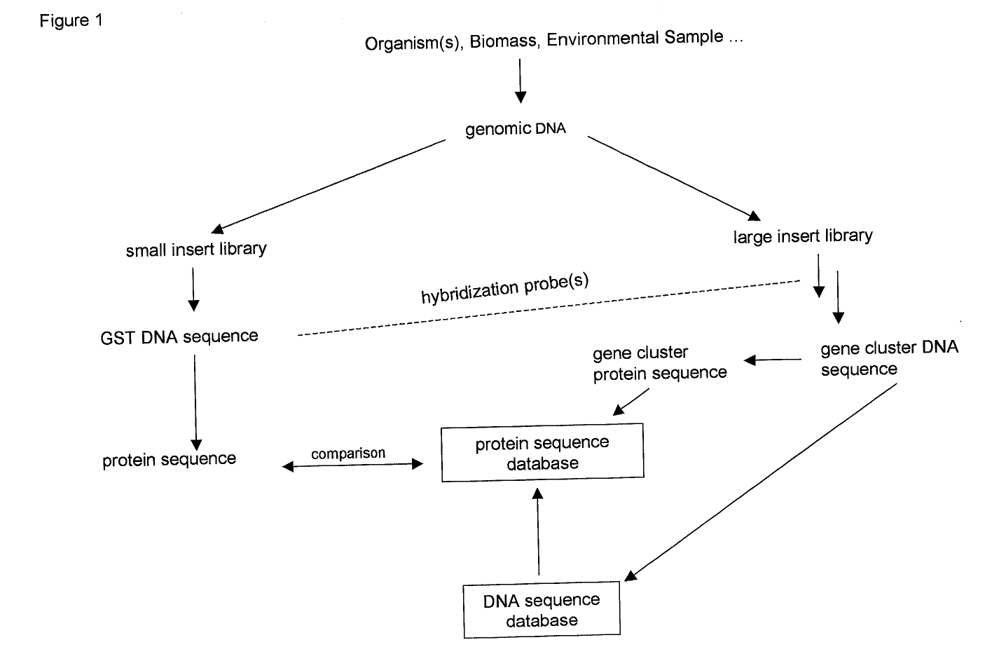

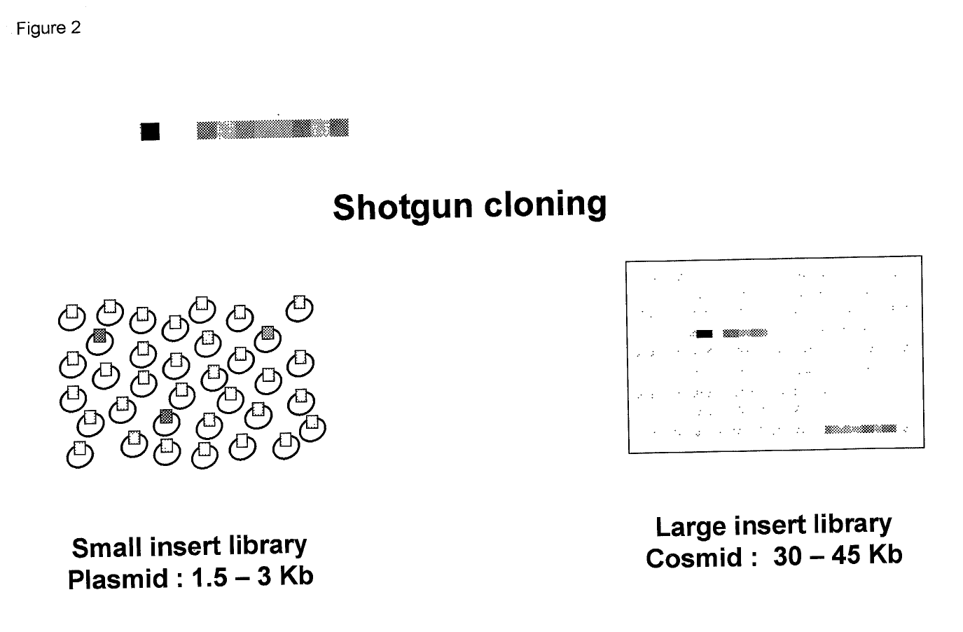

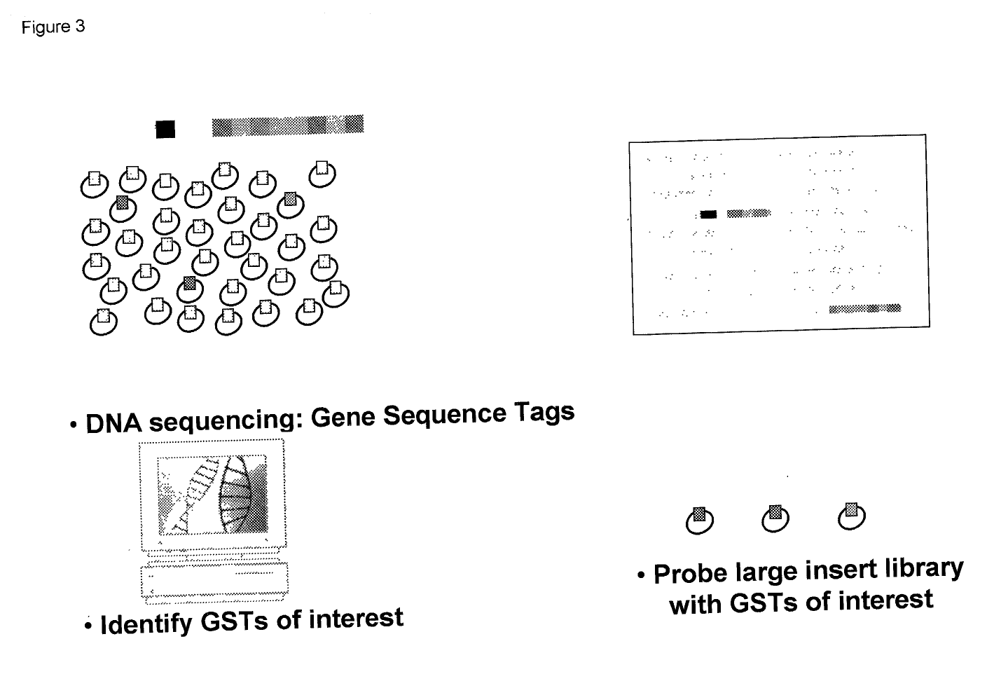

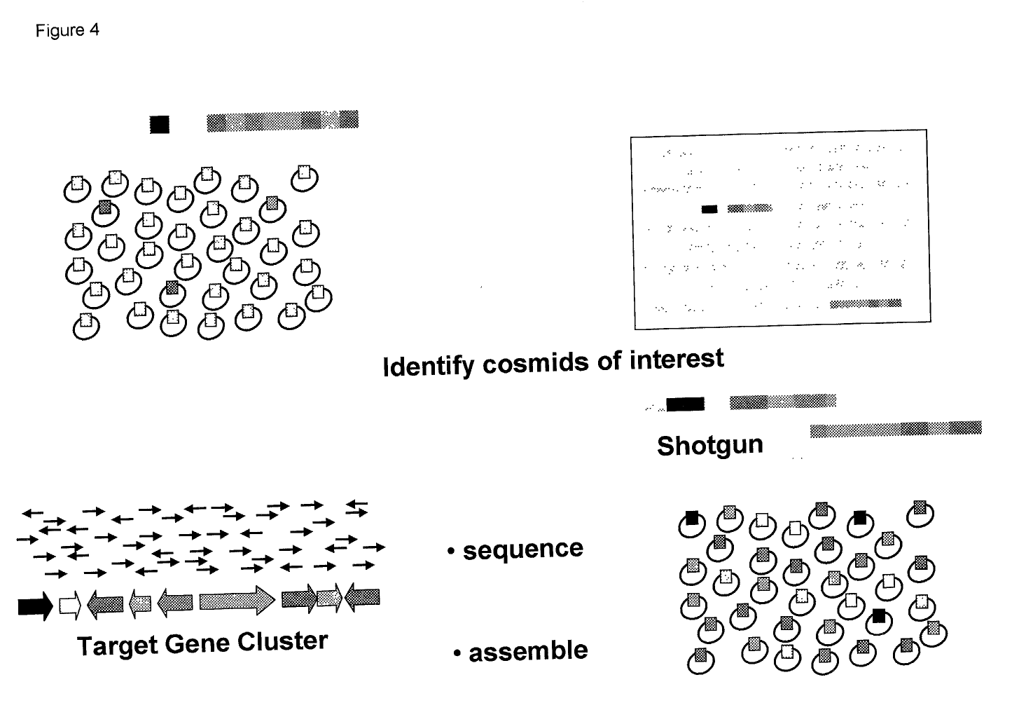

Reviewing the method by reference to the figures, high molecular weight genomic DNA of interest is isolated from a cell mass or biomass (FIG. 1). A small insert library and a large insert library are constructed by a shotgun cloning approach so as to contain randomly generated fragments of DNA (FIG. 2). The small insert library is composed of about 500 individual clones each containing a piece of genomic DNA insert in the range of 1.5-3 kb carried on a cloning vector that can be propagated in a suitable host organism. The large insert library is composed of an appropriate number of individual clones each containing a piece of genomic DNA of interest that is at least 30 kb carried on a cloning vector that can be propagated in a suitable host organism. The small insert library serves as a genomic DNA sampling that is sequenced to generate Gene Sequence Tags (GSTs) as illustrated in FIG. 3. Computer-assisted analysis of the GSTs identifies those GSTs likely to reside within the target gene cluster (GSTs of interest). Molecular probe(s) are then designed from the GSTs of interest and are used to identify, by nucleic acid hybridization, the clones in the large insert library that contain the probe(s). Once identified, the large insert clone(s) of interest are sequenced by a shotgun method similar to that employed on genomic DNA for the generation of the small insert library (FIG. 4). A sufficient number of shotgun sequences are done so as to allow for computer-assisted reconstruction or assembly of the entire sequence of the large insert clone(s).

It is to be understood that the embodiments described herein are for illustrative purposes only and that various modifications or changes in light thereof will be suggested to persons skilled in the art and are to be included within the spirit and purview of this application and scope of the appended claims. All publications, patents and patent applications cited herein are hereby incorporated by reference in their entirety for all purposes.

The following examples use many techniques well known and accessible to those skilled in the art. Enzymes are obtained from commercial sources and are used according to the vendors' recommendations or other variations known to the art. Abbreviations and nomenclature are employed as commonly used in professional journals such as those referred to herein.

### EXAMPLES cl Example 1

Identification of the Ramoplanin Biosynthetic Locus in Actinoplanes sp. ATCC 33076.

Actinoplanes sp. ATCC 33076 was previously shown to naturally produce ramoplanin, a biologically active lipodepsipeptide (U.S. Pat. No. 4,303,646). The genetic locus involved in the production of this compound was not previously identified.

Actinoplanes sp. strain ATCC 33076 was obtained from the American Tissue Culture Collection (ATCC) and cultured according to standard microbiological techniques (Kieser et al., supra). Confluent mycelia from oatmeal agar plates were used for the extraction of genomic DNA as previously described (Kieser et al., supra) and the size range of the DNA obtained was assessed on agarose gels by electrical field inversion techniques as described by the manufacturer (FIGE, BioRad). The DNA serves for the preparation of a small size fragment genomic sampling library (GSL),i.e. the small insert library, as well as a large size fragment cluster identification library (CIL), i.e. the large insert library. Both libraries contained DNA fragments generated randomly from genomic DNA and, therefore, they represented the entire genome of Actinoplanes sp.

For the generation of the GSL library, genomic DNA was randomly sheared by sonication. DNA fragments having a size range between 1.5 and 3 kb were fractionated on a agarose gel and isolated using standard molecular biology techniques (Sambrook et al., supra). The ends of the obtained DNA fragments were repaired using T4 DNA polymerase (Roche) as described by the supplier. This enzyme creates DNA fragments with blunt ends that can be subsequently cloned into an appropriate vector. The repaired DNA fragments were subcloned into a derivative of pBluescript SK+ vector (Stratagene) which does not allow transcription of cloned DNA fragments. This vector was selected as it contains a convenient polylinker region surrounded by sequences corresponding to universal sequencing primers such as T3, T7, SK, and KS (Stratagene). The unique EcoRV restriction site found in the polylinker region was used as it allows insertion of blunt-end DNA fragments. Ligation of the inserts, use of the ligation products to transform E. coli DH10B host and selection for recombinant clones were performed as previously described (Sambrook et al., supra). Plasmid DNA carrying the Actinoplanes sp. genomic DNA fragments was extracted and the insert size of 1.5 to 3 kb was confirmed by electrophoresis on agarose gels. Using this procedure a library of small size random genomic DNA fragments is generated that covers the entire genome of the studied microorganism. The number of individual clones that can be generated is infinite but only a small number is further analyzed to sample the microorganism's genome.

To generate the CIL library, high molecular weight genomic DNA was partially digested with a frequent cutting restriction enzyme, Sau3A (G|ATC). This enzyme generates random fragments of DNA ranging from the initial undigested size of the DNA to short fragments of which the length is dependent upon the frequency of the enzyme DNA recognition site in the genome and the extent of the DNA digestion. Conditions generating DNA fragments having an average length of ˜40 kb were chosen (Sambrook et al., supra). The Sau3A restricted DNA was ligated into the BamHI site of the SuperCos-1 cosmid cloning vector (Stratagene) and packaged into phage particles (Gigapack III XL, Stratagene) as specified by the supplier. E. coli strain DH10B was used as host and 864 recombinant clones carrying cosmids were selected and propagated to generate the CIL library. Considering an average size of 8 Mb for a streptomyces genome and an average size of 35 kb of genomic insert in the CIL library, this library represents a 4-fold coverage of the microorganism's entire genome. Subsequently, the Actinoplanes sp. CIL library was transferred onto membrane filters (Schleicher & Schnell) as specified by the manufacturer.

The GSL library was analyzed by sequence determination of the cloned genomic DNA inserts. The universal primers KS or T7, referred to as forward (F) primer, were used to initiate polymerization of labeled DNA. Extension of at least 700 bp from the priming site can be routinely achieved using the TF, BDT v2.0 sequencing kit as specified by the supplier (Applied Biosystems). Sequence analysis of the generated fragments (Genomic Sequence Tags, GSTs) was performed using a 3700 ABI capillary electrophoresis DNA sequencer (Applied Biosystems). The average length of the DNA sequence reads was ˜700 bp. Further analysis of the obtained GSTs was performed by sequence homology comparison to various protein sequence databases. The DNA sequences of the obtained GSTs were translated into amino acid sequences and compared to the National Center for Biotechnology Information (NCBI) nonredundant protein database and the proprietary Ecopia natural product biosynthetic gene Decipher™ database using previously described algorithms (Altschul et al., supra). Sequence similarity with known proteins of defined function in the database enables one to make predictions on the function of the partial protein that is encoded by the translated GST.

A total of 882 Actinoplanes sp. GSTs were generated and analyzed by sequence comparison. Sequence alignments displaying an E value of at least e-5 were considered as significantly homologous and retained for further evaluation. GSTs showing similarity to a gene of interest can be at this point selected and used to identify larger segments of genomic DNA including the gene of interest. Ramoplanins produced by Actinoplanes sp. belong to the family of polypeptide antibiotics. Polypeptides are synthesized by nonribosomal peptide synthase (NRPS) enzymes that perform a series of condensations and modifications of aminoacids. Many members of this enzymatic class are found in protein databases rendering possible the identification of an unknown NRPS by sequence similarity. Analysis of the Actinoplanes sp. GSTs revealed the presence of 3 GSTs having similarity to known NRPS proteins in the NCBI nonredundant protein database (Table 1). The obtained E values confirm that these GSTs encode partial NRPS sequences. The 3 NRPS GSTs were selected for the generation of oligonucleotide probes which were then used to identify gene clusters harboring the specific NRPS genes in the CIL library.

Oligonucleotide probes were designed from the nucleotide sequence of the selected GSTs, radioactively labeled, and hybridized to the CIL library using standard molecular biology techniques (Sambrook et al., supra, Schleicher & Schnell). Positive clones were identified, cosmid DNA was extracted (Sambrook et al., supra) and entirely sequenced using a shotgun sequencing approach (Fleischmann et al., Science, 269:496-512). Identification of the original GSTs, used to generate the oligonucleotide probes, within the DNA sequence of the obtained cosmids proved that these cosmids indeed carried the gene cluster of interest.

Generated sequences were assembled using the Phred-Phrap algorithm (University of Washington, Seattle, USA) recreating the entire DNA sequence of the cosmid insert. Reiterations of hybridizations of the CIL library with probes derived from the ends of the original cosmid allow indefinite extension of sequence information on both sides of the original cosmid sequence until the complete sought-after gene cluster is obtained. Application of this method on Actinoplanes sp. and use of the above-described NRPS GST probes yielded 6 cosmids. Complete sequence of these cosmids and analysis of the proteins encoded by them undoubtedly demonstrated that the gene cluster obtained was indeed responsible for the production of ramoplanin. Subsequent inspection of the ramoplanin biosynthetic cluster sequence (˜80 kb) revealed the presence of 3 additional GSTs from the GSL library, bringing the total number of ramoplanin locus GSTs to 6. The genetic locus responsible for the biosynthesis of ramoplanin and identified according to the present invention is disclosed in U.S. Ser. No. 60/239,924 filed on Oct. 13, 2000.

TABLE 1

| | Length | Proposed | | | Proposed function of |
|---|---|---|---|---|---|
| | (bp) | function | Homology | Probability | protein match |
| GST1 | 632 | NRPS | PIR T36248 | 3.00^{E}−20 | CDA peptide synthetase |
| | | | | | I in Streptomyces |
| | | | | | coelicolor |
| GST2 | 592 | NRPS | PIR T36248 | 5.00^{E}−28 | CDA peptide synthetase |
| | | | | | I in Streptomyces |
| | | | | | coelicolor |
| GST3 | 502 | NRPS | PIR T36180 | 7.00^{E}−31 | CDA peptide synthetase |
| | | | | | III in Streptomyces |
| | | | | | coelicolor |

### Example 2

Identification of a New Natural Product Biosynthetic Locus in Streptomyces mobaraensis.

Streptomyces mobaraensis was previously shown to naturally produce a variety of biologically active compounds including piericidins, pactamycin, and detoxins (Tamura et al., 1963, Agr. Biol. Chem., Vol. 27, No. 8, pp. 576-582). The genetic loci responsible for the production of these compounds and, hence, the enzymatic mechanisms involved in their biosynthesis have not been previously characterized.

Streptomyces mobaraensis strain NRRL B-3729 was obtained from the Agricultural Research Service collection (ARS) and cultured according to standard microbiological techniques (Kieser et al., supra). All subsequent experimental procedures were performed as described in Example 1.

A total of 450 GSTs were generated and analyzed by sequence comparison. Among these GSTs, two showed similarity to enzymes involved in deoxysugar biosynthesis (Table 2). There are several classes of natural compounds such as macrolides, polypeptides, anthracyclines, enediynes, polyenes that are glycosylated with typical and/or unusual glycosyl groups. Other metabolites such as orthosomycins and aminoglycosides are mainly composed of modified deoxysugar moieties (Weymouth-Wilson, The role of carbohydrates in biologically active natural products, Nat. Prod. Rep., 1997, 99-110). Specific enzymes are required for the biosynthesis of unusual sugars from natural sugar precursors as well as glycosyltransferase enzymes that catalyze the transfer of the sugar to a specific backbone structure (Liu and Thorson, Pathways and mechanisms in the biogenesis of novel deoxysugars by bacteria, Annu. Rev. Microbiol., 48: 223-256). The presence of two sugar biosynthetic genes in Streptomyces mobaraensis is of interest as the natural products shown to be produced by this microorganism do not contain any sugar residue. GST1 was used to probe the S. mobaraensis CIL library as above-described (Example 1).

Positive clones were identified and sequenced. The original GST1 was identified within the sequenced cosmid. One reiteration of the same method were applied providing two overlapping cosmids covering the entire biosynthetic cluster. Analysis of the proteins encoded by this cluster demonstrated the presence of a novel biosynthetic locus (˜45 kb) having the potential to produce an avilamycin-like compound, member of the orthosomycin group of antibiotics composed of a series of deoxysugar residues. The genetic locus responsible for the biosynthesis of this avilamycin-like compound and identified according to the present invention is disclosed in U.S. Ser. No. 60/279,709 filed on Mar. 30, 2001.

TABLE 2

| | Length | Proposed | | | Proposed function of |
|---|---|---|---|---|---|
| | (bp) | function | Homology | Probability | protein match |
| GST1 | 738 | sugar dehydratase | PIR T30873 | 2.00^{E}−74 | dNDP-glucose |
| | | | | | dehydratase in |
| | | | | | Streptomyces |
| | | | | | viridochromogenes |
| GST2 | 601 | glycosyltransferase | PIR F75099 | 2.00E−05 | rhamnosyl transferase in |
| | | | | | Pyrococcus abyssii |

### Example 3

Identification of the Anthramycin Producing Biosynthetic Locus in Streptomyces refuineus.

Streptomyces refuineus var. thermotolerans was shown to produce a benzodiazepine antibiotic, anthramycin, that covalently binds to the minor groove of DNA. Anthramycin has been shown to possess various potent biological activities including antibiotic, antitumor and antiviral activities. The biosynthetic locus responsible for the production of anthramycin was not previously characterized.

Streptomyces refuineus var. thermotolerans strain NRRL-3143 was obtained from the Agricultural Research Service collection (ARS) and cultured using standard microbiological techniques (Kieser et al., supra). Subsequent experimental procedures for cloning and analyzing the genetic material of this microorganism were as described in Example 1. A total of 486 GSTs were analyzed by sequencing and protein homology comparison to the NCBI protein database and the Ecopia Decipher™ proprietary protein database. Precursor feeding studies have established two distinct moieties in the anthramycin molecule that derive from tryptophan via the kynurenine pathway and catabolism of L-tyrosine (Hurley et al., 1975). The two modified amino acids are linked together through an amide bond typically catalyzed by nonribosomal peptide synthases (NRPS). Analysis of the S.refuineus GSTs revealed the presence of a GST showing aminoacid similarity to an alpha-aminoadipate reductase protein in Candida albicans, enzyme that has a domain organization similar to these of NRPSs (Table 3). This GST was subsequently used to probe the S. refuineus CIL library as described in Example 1.

Cosmids positive by hybridization were obtained and analyzed by sequence determination. The presence of the original GST that was used to screen the CIL library was determined in the sequenced cosmid confirming that this cosmid carried the sought-after gene cluster. After one reiteration of the described method, two overlapping cosmids covering the entire anthramycin biosynthetic locus were obtained. Analysis of the genetic information derived from these two cosmids clearly demonstrated the presence and defined the boundaries of the anthramycin biosynthetic locus (˜33 kb). The genetic locus responsible for the biosynthesis of anthramycin and identified according to the present invention is disclosed in U.S. Ser. No. 60/296,744 filed on Jun. 11, 2001.

TABLE 3

| | Length | Proposed | | | Proposed function of |
|---|---|---|---|---|---|
| | (bp) | function | Homology | Probability | protein match |
| GST1 | 426 | reductase | gb | 2.00E−06 | alpha-aminoadipate |
| | | | AAC02241.1 | | reductase in Candida |
| | | | | | albicans |

### Example 4

Identification of the Micromonospora carbonacea Everninomycin Biosynthetic Pathway.

Everninomycins are oligosaccharide antibiotics that are members of the orthosomycin chemical class. This class is characterized by the presence of orthoester groups joining, together with glycosidic linkages, various deoxysugar residues. Everninomycins are produced by several variants of the microorganism Micromonospora carbonacea (Weinstein et al., Antimicrobial Agents and Chemotherapy—1964, 24-32, 1964; U.S. Pat. No. 3,499,078).

The Agricultural Research Service collection (ARS) Micromonospora carbonacea subsp. aurantiaca strain NRRL 2997 was used to determine the everninomycin biosynthetic locus. All experimental procedures were as described (Example 1). The presence of several deoxysugar residues in the chemical structure everninomycins is a clear indication that well-described enzymatic activities involved in the generation of these unusual sugar residues should participate in the biosynthesis of these compounds. Analysis of the M. carbonacea genome sampling library (GSL), of a total of 437 GST, revealed the presence of two GSTs having sequence homology to enzymes involved in the synthesis of deoxysugar residues from natural sugar precursors (Table 4).

Both GSTs were used as probes for screening the Micromonospora carbonacea cluster identification library (CIL). Overlapping cosmids positive for both probes were obtained suggesting a near proximity for the two GSTs in the gene cluster. Analysis of sequenced cosmids revealed the presence of the original GSTs confirming that the obtained gene cluster was indeed the targeted one. After two reiterations of this method, 3 overlapping cosmids were obtained.

DNA sequence determination of these cosmids and analysis of the encoded proteins by sequence similarity undoubtedly established this locus as the one responsible for the biosynthesis of everninomycin. Additional DNA sequence inspection of the everninomycin locus (˜58 kb) showed that a total of 7 GSTs obtained from the original screening of the GSL library, including the ones that were used to probe the CIL library, were part of the everninomycin locus. The genetic locus responsible for the biosynthesis of everninomycin, identified according to the present invention, is disclosed in U.S. Ser. No. 09/769,734 filed on Jan. 27, 2001.

TABLE 4

| | Length | Proposed | | | Proposed function of |
|---|---|---|---|---|---|
| | (bp) | function | Homology | Probability | protein match |
| GST1 | 787 | sugar | PIR T30873 | 6.00^{E}−90 | dNDP-glucose |
| | | dehydratase | | | dehydratase in |
| | | | | | Streptomyces |
| | | | | | viridochromogenes |
| GST2 | 601 | dNTP-sugar | PIR T30872 | 9.00^{E}−38 | dNDP-glucose synthase |
| | | synthase | | | in Streptomyces |
| | | | | | viridochromogenes |

### Example 5

Identification of a phiC31-like Prophage in Streptomyces aizunensis NRRL B-11277

Streptomyces aizunensis NRRL B-11277 was obtained from the Agricultural Research Service collection (ARS) and cultured according to standard microbiological techniques (Hopwood). Unless otherwise stated, all subsequent experimental procedures were performed as described in Example 1.

A total of 462 GSTs were generated and analyzed by sequence comparison. Three GSTs showed similarity to genes from the actinophage phiC31 (Smith et al., The complete genome sequence of the Streptomyces temperate phage phiC31: evolutionary relationships to other viruses) (Table 5). Prophages are integrated versions of the genome of bacterial viruses and hence represent a type of gene cluster; that is, they include a collection of closely linked genes whose function is to propagate progeny virions. Oligonucleotide probes based on the three GSTs and probed a S. aizunensis cosmid library were designed.

Several positive cosmid clones were identified and among these two non-overlapping clones were selected for further sequencing analysis as described in Example 1. Cosmid 1 consisted of a 35 kb insert that included the sequences of both GST 1 and GST 2. Interestingly, the GST1 and GST 2 sequences (in the context of the insert of cosmid 1) were flanked by sequences encoding several other phiC31-like genes, and most notably these include the “late” genes of phiC31. Cosmid 1 also included a short sequence with significant similarity to the Cos sites of phage phiC31 and contained tRNA sequences in close proximity to this Cos-site-like element. Cosmid 2 consisted of an insert of at least 32 kb that included the sequences of GST 3. As expected, the GST 3 sequences (in the context of the insert of cosmid 2) were flanked by sequences encoding several other phiC31-like genes, and most notably these include the “early” genes of phiC31. Thus, a phiC31-like prophage was identified within the genome of S. aizunensis.

TABLE 5

| | Length | Proposed | | | Proposed function of |
|---|---|---|---|---|---|
| | (bp) | function | Homology | Probability | protein match |
| GST1 | 501 | terminase large | CAA07103 | 2.00^{E}−66 | phiC31 gp33; |
| | | subunit | | | terminase, large subunit |
| GST2 | 501 | protease | CAA07105 | 7.00^{E}−41 | phiC31 gp35; protease |
| GST3 | 501 | primase/helicase | CAA07134 | 1.00^{E}−58 | phiC31 gp9a; |
| | | | | | primase/helicase |

1. A method for detecting genes which act together in a coordinated manner and are clustered together in a genome comprising:
a. preparing from isolated genomic DNA a small insert library of DNA fragments of the genomic DNA and a large insert library of DNA fragments of the genomic DNA;
b. determining the DNA sequence of at least part of the fragments in the small insert library to form a plurality of Gene Sequence Tags (GSTs);
c. comparing, under computer control, the DNA sequence of the GSTs or the amino acid sequences corresponding to the DNA sequence of the GSTs with a database containing genes, gene fragments or DNA, or amino acid sequences known to be part of a cluster of genes that act together in a coordinated manner and are clustered together on a chromosome to identify GSTs that have similar structure to genes, gene fragments or amino acid sequences known to be part of a cluster of genes that act together in a coordinated manner; and
d. using the GSTs having homology to genes, gene fragments or amino acid sequences known to be part of a cluster of genes that act together in a coordinated manner as probes to screen the large insert library of genomic DNA to detect genes which act together in a coordinated manner and are clustered together on a chromosome.

2. The method according to claim 1 further comprising:
e. determining the sequence of the large insert fragments from step d).

3. The method according to claim 1 wherein step b) further comprises the additional step of translating the DNA sequence of the GSTs to generate a corresponding amino acid sequence, and wherein in step c) comparing is done on the basis of amino acid sequences that correspond to genes or gene fragments known to be part of a cluster of genes that act together in a coordinated manner and are clustered together.

4. The method according to claim 1 wherein in step c) the identification of GSTs that have similar structure to genes, gene fragments of amino acid sequences known to be part of a cluster of genes that act together in a coordinated manner is done by computer assisted homology analysis.

5. The method according to claim 1 wherein the genomic DNA is obtained from a microorganism.

6. The method according to claim 1 wherein the genomic DNA is obtained from a natural habitat or biomass.

7. The method according to claim 1 wherein the DNA fragments in the small insert library are between about 1.5 kbp and about 10 kbp.

8. The method according to claim 7 wherein the DNA fragments in the small insert library are between about 1.5 kbp and about 5 kbp.

9. The method according to claim 1 wherein the DNA fragments in the large insert library range between about 10 kbp and several hundreds of kilobases depending upon the cloning vehicle used.

10. The method according to claim 9 wherein the DNA fragments in the large insert library are between about 30 kbp to about 50 kbp.

11. A method for identifying genes and gene clusters involved in the biosynthesis of microbial natural products comprising:
a. isolating genomic DNA from an organism or natural environment;
b. preparing a small insert library of DNA fragments of about 1.5 kbp to about 10 kbp of the genomic DNA, and a large insert library of DNA fragments of the genomic DNA;
c. determining the sequence of at least part of the fragments in the small insert library to form a plurality of gene sequence tags (GSTs);
d. comparing, under computer control, the sequences of the GSTs with a database containing genes, gene fragments or amino acid sequences known to be involved in the biosynthesis of microbial natural products to identify by sequence homology the GSTs that have a similar structure to genes, gene fragments or amino acid sequences known to be involved in the biosynthesis of microbial natural products; and
e. using the GSTs having similar structure to genes, gene fragments or amino acid sequences known to be involved in the biosynthesis of microbial natural products, or portions thereof, as probes to screen the large insert library of genomic DNA to detect gene clusters involved in the biosynthesis of microbial natural product.

12. The method according to claim 11 wherein step c) further comprises the additional step of translating the DNA sequence of the GSTs to generate a corresponding amino acid sequence, and step c) comprises comparing, under computer control, the amino acid sequences of the GSTs with amino acid sequences corresponding to genes or gene fragments known to be involved in the biosynthesis of microbial natural products.

13. The method of claim 11 wherein the DNA fragments in the small insert library are between about 1.5 kbp to about 3kbp.

14. The method of claim 11 wherein the DNA fragments in the large insert library are between about 30 kbp and about 50 kbp.

15. A method for cloning gene clusters involved in the biosynthesis of microbial natural products comprising:
a. isolating genomic DNA from an organism or natural environment;
b. preparing a small insert library of DNA fragments of about 1.5 kbp to 10 kbp of the genomic DNA, and a large insert library DNA fragments of the genomic DNA;
c. sequencing at least part of the fragments in the small insert library to form a plurality of gene sequence tags (GSTs);
d. comparing, under computer control, the sequences of the GSTs with a database containing genes, gene fragments, or amino acid sequences known to be involved in the biosynthesis of microbial natural products to identify the GSTs that have a similar structure to genes, gene fragments or amino acid sequences known to be involved in the biosynthesis of microbial natural products;
e. using the GSTs having similar structure to genes, gene fragments or amino acid sequences known to be involved in the biosynthesis of microbial natural products as probes to screen the large insert library of genomic DNA to detect gene clusters involved in the biosynthesis of microbial natural product; and
f. determining the DNA sequence of the large insert genomic DNA detected in step e).
g. The method according to claim 15 wherein step c) further comprises the additional step of translating the DNA sequence of the GSTs to generate a corresponding amino acid sequence, and step c) comprises comparing, under computer control, the amino acid sequences of the GSTs with amino acid sequences corresponding to genes or gene fragments known to be involved in the biosynthesis of microbial natural products.

16. The method according to claim 15 wherein step c) further comprises the additional step of translating the DNA sequence of the GSTs to generate a corresponding amino acid sequence, and step d) comprises comparing, under computer control, the amino acid sequence of the GSTs with amino acid sequences corresponding to genes or gene fragments known to be involved in the biosynthesis of microbial natural products.

17. The method according to claim 15 wherein step f) involves determining the sequence of the large insert fragments from step d) by way of shotgun DNA sequencing.

18. The method according to claim 15 wherein step f) comprises determining the sequence of the large insert fragments from step d) by a technique selected from a subcloning technique, a primer walking technique, or a nested deletion technique.

19. The method of claim 15 wherein in step d) the identification of GSTs that have similar structure to genes, gene fragments or amino acid sequences known to be that have a similar structure to genes or gene fragments known to be involved in the biosynthesis of microbial natural products is done by computer assisted homology analysis.

20. The method of claim 15 wherein the DNA fragments in the small insert library are between about 1.5 kbp and about 3 kbp, and the DNA fragments in the large insert library are between about 30 kbp and about 50 kbp.
